(19) United States
(12) Patent Application Publication
(10) Pub. No.: US 2013/0202706 A1
(43) Pub. Date: Aug. 8, 2013

(54) NANOSTRUCTURED ATORVASTATIN, ITS PHARMACEUTICALLY ACCEPTABLE SALTS AND COMPOSITIONS OF THEM, PROCESS FOR THE PREPARATION THEREOF AND PHARMACEUTICAL COMPOSITIONS CONTAINING THEM

(71) Applicants: Genovéva Filipcsei, Budapest (HU); Zsolt Ötvös, Csongrad (HU); Gábor Heltovics, Budapest (HU); Ferenc Darvas, Budapest (HU)

(72) Inventors: Genovéva Filipcsei, Budapest (HU); Zsolt Ötvös, Csongrad (HU); Gábor Heltovics, Budapest (HU); Ferenc Darvas, Budapest (HU)

(73) Assignee: DRUGGABILITY TECHNOLOGIES IP HOLDCO (JERSEY) LTD., St. Helier, Jersey (GB)

(21) Appl. No.: 13/702,349

(22) PCT Filed: Jun. 8, 2011
(86) PCT No.: PCT/HU2011/000051

§ 371 Date: Jan. 10, 2013

(30) Foreign Application Priority Data
Jun. 8, 2010 (HU) P1000299

(51) Int. Cl. A61K 9/14

(52) U.S. Cl. CPC A61K 9/14
USPC 424/489; 514/423

(57) ABSTRACT

The present invention is directed to nanostructured Atorvastatin, its pharmaceutically acceptable salts and compositions of them, process for the preparation thereof and pharmaceutical compositions containing them. The nanoparticles of Atorvastain, its pharmaceutically acceptable salts and compositions of them according to the invention have an average particle size of less than about 600 nm. The stable amorphous nanostructured particles of the present invention are characterized by increased solubility and bioequivalent biological performance compared to the marketed crystalline drug. Atorvastatin is a member of the drug class known as statins, used for lowering blood cholesterol. It also stabilizes plaque and prevents strokes through anti-inflammatory and other mechanisms.

### FIELD OF THE INVENTION

The present invention is directed to nanostructured Atorvastatin, its pharmaceutically acceptable salts and compositions of them, process for the preparation thereof and pharmaceutical compositions containing them.

The nanoparticles of Atorvastatin, its pharmaceutically acceptable salts and compositions of them according to the invention have an average particle size of less than about 600 nm. The stable amorphous nanostructured particles of the invention are presented by increased solubility and bioequivalent biological performance compared to the marketed crystalline drug. Atorvastatin is a member of the drug class known as statins, used for lowering blood cholesterol. It also stabilizes plaque and prevents strokes through anti-inflammatory and other mechanisms.

### BACKGROUND OF THE INVENTION

A. Background Regarding to Nanoparticle Formation/Production

Nowadays, the active ingredient developers run out of new chemical entities with high solubility; most compounds that are approved or enter development processes are poorly soluble and/or have low permeability. The traditional approaches to increase the solubility and dissolution rate of these compounds are very limited. Chemical modification, like salt- or prodrug formation and inclusion of ionizable groups could result in higher performance of the active compounds. However, these structural modifications can lead to inactivity or instability of the active compounds in many cases. Conventional solid or liquid formulations (e.g.; micronization, milling, solid dispersion, liposomes) could also be useful tools for the researchers to increase the solubility of the compounds, but the efficiency of the formulation is far behind the chemical modification. Nevertheless, these conservative approaches are very time- and cost-consuming procedures with high failure rates.

Nanoformulation is currently one of the most progressive fields of the pharmaceutical industry to increase solubility, bioavailability as well as reduce food and side effects of such active ingredients.

Nanoformulation is the reduction of particles size down to below 200 nm. The reduction of particle size leads to significantly increased dissolution rate of the active ingredients, which in turn can lead to increases in bioavailability.

There are two main approaches to making nanoparticles: “top-down” and “bottom-up” technologies. The conventional top-down approach basically relies on mechanical attrition to render large crystalline particles into nanoparticles. The bottom-up approach relies on controlled precipitation. The process involves dissolving the drugs in a solvent and precipitation in a controlled manner to nanoparticles through addition of an antisolvent.

Technologies relying on milling (top-down) or high-pressure homogenization (mixture of uncontrolled-bottom-up and top-down) are cost and time consuming methods. Both processes require high energy. This means that a large number of active compounds cannot be nanoformulated with these approaches due to heat induced active form conversion. For example, salt or active compounds with low melting point cannot be milled or high-pressure homogenized. The scale-up (industrial applicability) of the high energy processes are difficult and limited in many cases. These technologies target only late stage formulation or reformulation of poorly soluble active compounds to improve their efficiency.

Drug nanoparticles can be made using, for example, milling, homogenization, precipitation techniques, or supercritical fluid techniques, as known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, U.S. Pat. No. 5,534,270, U.S. Pat. No. 5,510,118, U.S. Pat. No. 5,470,583, US Pat. Application 2009/0028948 and EP 1658053.

B. Background Regarding Atorvastatin

Atorvastatin is an inhibitor of 3-hydroxy-3-methylglutaryl-coenzyme A (HMG-CoA) reductase. This enzyme catalyzes the conversion of HMG-CoA to mevalonate, an early and rate-limiting step in cholesterol biosynthesis. It is marketed in its calcium salt by Pfizer under the trade name Lipitor.

Atorvastatin calcium is [R—(R*, R*)]-2-(4-fluorophenyl)-β, δ-dihydroxy-5-(1-methylethyl)-3-phenyl-4-[(phenylamino)carbonyl]-1Hpyrrole-1-heptanoic acid, calcium salt (2:1) trihydrate. The empirical formula of Atorvastatin calcium is (C33H34 FN2O5)2Ca.3H2O and its molecular weight is 1209.42. Its structural formula is:

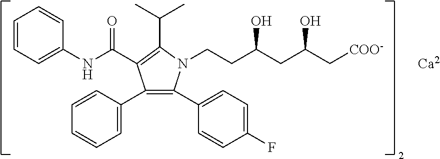

Atorvastatin calcium is a white to off-white crystalline powder that is insoluble in aqueous solutions of pH 4 and below. Atorvastatin calcium is very slightly soluble in distilled water, pH 7.4 phosphate buffer, and acetonitrile; slightly soluble in ethanol; and freely soluble in methanol.

LIPITOR tablets for oral administration contain 10, 20, 40, or 80 mg Atorvastatin and the following inactive ingredients: calcium carbonate, USP; candelilla wax, FCC; croscarmellose sodium, NF; hydroxypropyl cellulose, NF; lactose monohydrate, NF; magnesium stearate, NF; microcrystalline cellulose, NF; Opadry White YS-1-7040 (hypromellose, polyethylene glycol, talc, titanium dioxide); polysorbate 80, NF; simethicone emulsion.

Atorvastatin and its salts compositions are described, for example, in WO/2008/106901, WO/2002/059087, WO/2009/007856, WO/2003/068191, WO 2006/054308, WO/2008/082124, WO/2007/058664, WO/2008/039894, WO/2000/071116, WO/2004/110431, and nanoparticle of Atorvastatin Calcium is described for example, in WO/2006/125496, WO/2008/065502, WO/2009/075401, WO/2009/010842, WO/2008/135828, WO/2007/136830, WO/2008/032327, WO/2008/016837, WO/2007/069827, WO/2007/016306, WO/2008/002568, WO/2007/076295.

Atorvastatin is an “unusual” molecule that exists in a large number of polymorphs with various “amorphous” forms and a greater number of different crystalline forms but that Form I crystalline Atorvastatin calcium trihydrate is the active ingredient in Lipitor and the only form ever marketed by Pfizer. The originator also stated that the original Atorvastatin calcium utilized by Pfizer in early development was an amorphous solid. Pfizer then points out the differences between the amorphous and crystalline Atorvastatin, including chemical stability, impurities, particle size and dissolution profiles. Pursuit of competitiveness is sometimes challenging because the amorphous state is a non-equilibrium state; an amorphous active compound may crystallise during storage or during a manufacturing process. Atorvastatin (Lipitor®) was formulated as an amorphous salt but it was observed to crystallise during phase III clinical trials. This drawback delayed the launching of the drug onto the market and pointing out the weakness of the production procedure and the amorphous formula produced by the originator's method described in the U.S. Pat. No. 5,969,156. There are several patents or methods for preparing amorphous Atorvastatin from different salts, e.g.: WO/2001/028999, WO/2006/039441, WO/2006/046109, WO/2005/073187, WO/2000/071116, WO/2004/085391, WO/2003/016317.

Pharmacological Properties

Atorvastatin is rapidly absorbed after oral administration; maximum plasma concentrations occur within 1 to 2 hours. Extent of absorption increases in proportion to Atorvastatin dose. The absolute bioavailability of Atorvastatin (parent drug) is approximately 14% and the systemic availability of HMG-CoA reductase inhibitory activity is approximately 30%. The low systemic availability is attributed to presystemic clearance in gastrointestinal mucosa and/or hepatic first-pass metabolism. Although food decreases the rate and extent of drug absorption by approximately 25% and 9%, respectively, as assessed by Cₘₐₓ and AUC, LDL-C reduction is similar whether Atorvastatin is given with or without food. Plasma Atorvastatin concentrations are lower (approximately 30% for Cₘₐₓ and AUC) following evening drug administration compared with morning. However, LDL-C reduction is the same regardless of the time of day of drug administration.

Because of the low solubility of Atorvastatin in water and its unstable amorphous formation, there is a need in the art to enhance the solubility/dissolution rate and increase the stability of the amorphous form to overcome on these and other problems associated with prior conventional Atorvastatin Calcium formulations. These problems can be solved by novel nanostructured particle formation characterized by increased solubility/dissolution rate and having stable amorphous form and bioequivalence with the marketed drug described in the present invention.

### DESCRIPTION OF THE INVENTION

The present invention describes nanostructured Atorvastatin, its pharmaceutically acceptable salts and compositions of them with increased solubility/dissolution rate and having stable amorphous formation and bioequivalence pharmacokinetic profile with the marketed drug.

The invention comprises novel nanostructured Atorvastatin and its pharmaceutically acceptable salts having an average particle size of less than about 600 nm.

Nanostructured Atorvastatin and its pharmaceutically acceptable salts according to the invention have an average particle size between 600 nm and 50 nm, preferably 400 nm and 50 nm, preferably 300 nm and 50 nm.

The invention further relates to a stable nanostructured Atorvastatin composition comprising:
(a) nanostructured Atorvastatin and its pharmaceutically acceptable salts having an average particle size of less than about 600 nm;
(b) at least one stabilizer and
(c) optionally any additional stabilizer for steric and electrostatic stabilization

wherein the composition of the invention is prepared preferably in a continuous flow reactor, more preferable in microfluidic based continuous flow reactor.

In the composition of the invention Atorvastatin or its pharmaceutically acceptable salt or co-crystal can be used in a phase selected from a crystalline phase, an amorphous phase, a semi-crystalline phase, a semi-amorphous phase and mixtures thereof.

As exemplified in the examples below, not every combination of stabilizers will result in a stable nanostructured particle formation. It was discovered, that stable nanostructured particles of Atorvastatin and its pharmaceutically acceptable salts can be made by continuous flow precipitation method using selected stabilizers.

The expression Atorvastatin is generally used for Atorvastatin and its pharmaceutically acceptable salts.

For the preparation of the composition of the invention stabilizers include nonionic, anionic, cationic, ionic polymers/surfactants and zwitterionic surfactants can be used. Combinations of more than one stabilizer can also be used in the invention. Useful stabilizers which can be employed in the invention include, but are not limited to known organic and inorganic pharmaceutical excipients. Such excipients include various polymers, low molecular weight oligomers, natural products, and surfactants.

Representative examples of stabilizers include hydroxypropyl methylcellulose, hydroxypropylcellulose, polyvinylpyrrolidone, sodium lauryl sulfate, gelatin, cetostearyl alcohol, polyethylene glycols, acetic acid ethenyl ester polymer with 1-ethenyl-2-pyrrolidinone (PVP/VA copolymers), sodium dodecyl benzene sulfonate, tocopheryl polyethylene glycol succinates, urea, citric acid, sodium-acetate, polyethoxylated castor oils and its derivateives, polyoxyethylene stearates, methylcellulose, hydroxyethylcellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide); poloxamines (e.g., Tetronic, also known as Poloxamine, which is a tetrafunctional block copolymer derived from sequential addition of propylene oxide and ethylene oxide to ethylenediamine, D-alfa-Tocopherol polyethylene glycol 1000 succinate, poly(2-ethyl-2-oxazoline), poly (methyl vinyl ether), random copolymers of vinyl pyrrolidone and vinyl acetate, such as Plasdone S630 and the like.

Examples of useful ionic stabilizers include, but are not limited to polymers, biopolymers, polysaccharides, cellulosics, alginates, phospholipids, nonpolymeric compounds, zwitterionic and ionic stabilizers, such as sodium lauryl sulfate and sodium dodecyl benzene sulfonate.

Advantages of the composition of the invention include, but are not limited to: (1) it has bioequivalent pharmacokinetic profile compared to the marketed drugs; (2) it has increased solubility of Atorvastatin and (3) increased rate of dissolution for Atorvastatin nanostructured particles as compared to conventional forms of the same active compound; (4) it is a stable amorphous formation of nanostructured Atorvastatin.

Another aspect of the invention is a process for the preparation of nanostructured Atorvastatin or its pharmaceutically acceptable salts comprising mixing an appropriate solvent of Atorvastatin or its pharmaceutically acceptable salt with a solution of one or more stabilizers if desired in the presence of a pharmaceutically acceptable acid in a continuous flow reactor, preferable in a microfluidic continuous flow reactor.

Preferably the process for the preparation of the composition of the invention is carried out by (1) dissolving Atorvastatin or its pharmaceutically acceptable salt and optionally one or more stabilizer in a suitable solvent; (2) adding the formulation from step (1) to a solution comprising at least one stabilizer; and (3) precipitating the formulation from step (2).

Preferably the process for the preparation of the composition of the invention is carried out by (1) dissolving Atorvastatin or its pharmaceutically acceptable salt and one or more stabilizer(s) in a suitable solvent;(2) adding the formulation from step (1) to a solution from step (1) to a solvent comprising optionally one or more stabilizer(s); and (3) precipitating the formulation from step (2).

The process is carried out by (a) using two different solvents miscible with each other, where Atorvastatin and its pharmaceutically acceptable salt is soluble only in one of them with the restriction that the applied stabilizer(s) is soluble in the solvents used. Such solvents may be dimethyl-sulfoxyde, methanol, ethanol, i-propanol, acetonitrile, tetrahydro-furane, acetone and pyridine preferably.

As a continuous flow reactor preferable a microfluidics based continuous flow reactor, described in the publication Microfluid Nanofluid DOI 10.1007/s10404-008-0257-9 by I. Hornyak, B. Borcsek and F. Darvas, is used.

The particle size of the nanostructured Atorvastatin may be influenced by the solvents used, the flow rate and the Atorvastatin—stabilizer ratio.

Another aspect of the invention is directed to the good/instantaneous redispersibility of solid amorphous nanostructured form of Atorvastatin in biologically relevant mediums, e.g.; physiological saline solution, pH=2.5 HCl solution.

Another aspect of the invention is a pharmaceutical composition comprising a stable nanostructured Atorvastatin or its pharmaceutically acceptable salts or composition of them according to the invention and optionally pharmaceutically acceptable auxiliary materials.

The pharmaceutical composition of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral administration in solid, liquid, local (powders, ointments or drops), or topical administration, and the like.

A preferred dosage form of the invention is a solid dosage form, although any pharmaceutically acceptable dosage form can be utilized.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols(propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alginates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and 0) lubricants, such as talc, calcium stearate, magnesium stearate, solid polyethylene glycols, sodium lauryl sulfate, or mixtures thereof For capsules, tablets, and pills, the dosage forms may also comprise buffering agents.

Besides such inert diluents, the composition can also include adjuvants, such as wetting agents, emulsifying and suspending agents, sweetening, flavoring, and perfuming agents.

The pharmaceutical compositions of the invention show enhanced lipophilicity/bioavailability/increased absorption and dissolution rate/reduced side effect, they can be used in a decreased dosage in used for lowering blood cholesterol and stabilizing plaque and preventing strokes through anti-inflammatory and other mechanisms.

The pharmaceutical compositions of the invention show increased solubility/dissolution rate and have stable amorphous formation and bioequivalent pharmacokinetic profile compared with the marketed drug.

A. Preferred Characteristics of the Atorvastatin Nanoparticles of the Invention

1. Increased Solubility and Dissolution Rate of Nanostructured Atorvastatin

Nanostructured Atorvastatin or its pharmaceutically acceptable salts compositions of the invention have increased solubility and dissolution profile due to the decreased particles size and nanostructured particle formation.

### EXAMPLE 1

Determination of Cₘₐₓ

The solubility of nanostructured Atorvastatin calcium compared to the reference API was determined in distillate water by UV-VIS measurements (Agilent 8453) at 241 nm wavelength and room temperature. The redispersed sample was filtered by 0.20 μm disposable syringe filter. In order to check the nanoparticle presence in the solution, it was irradiated by red laser pointer operating at 670 nm wavelength. If no scattering was observed the filtration was successful, the solution did not contain nanoparticles.

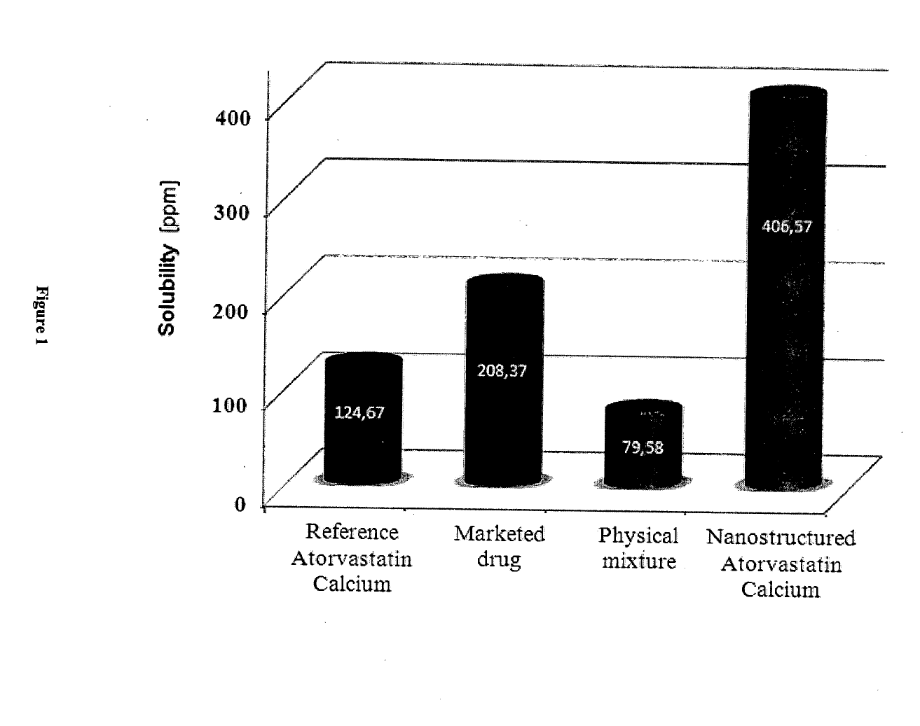

The solubility of the nanostructured Atorvastatin calcium is 0.40675 mg/mL (406.75 ppm) which is 3.26 times higher than the solubility of Atorvastatin calcium in distillate water as it seen in FIG. 1.

FIG. 1: Solubility Enhancement of Atorvastatin Calcium by Nanoformulation

### EXAMPLE 2

Comparative Dissolution Tests

Dissolution tests were performed by redispersing 2.5 mg reference Atorvastatin calcium and 13.7 mg nanostructured Atorvastatin calcium powder containing 2.5 mg Atorvastatin calcium in 5 mL distillate water. The suspension was stirred for 10 minutes, 1, 2, 4 and 24 hours and then it was filtered by 0.1 μm disposable syringe filter. Atorvastatin concentration was determined by UV-VIS spectrophotometer (Agilent 8453).

Due to the instantaneous redispersibility of nanostructured Atorvastatin calcium, more than 56% of the Atorvastatin content of the composition dissolves immediately upon the redispersion. Within 10 minutes the solution containing the redispersed nanostructured particles reaches its saturated state, the dissolved Atorvastatin content is 0.40675 mg/mL which is in a good correlation with the solubility of nanostructured Atorvastatin (FIG. 1.).

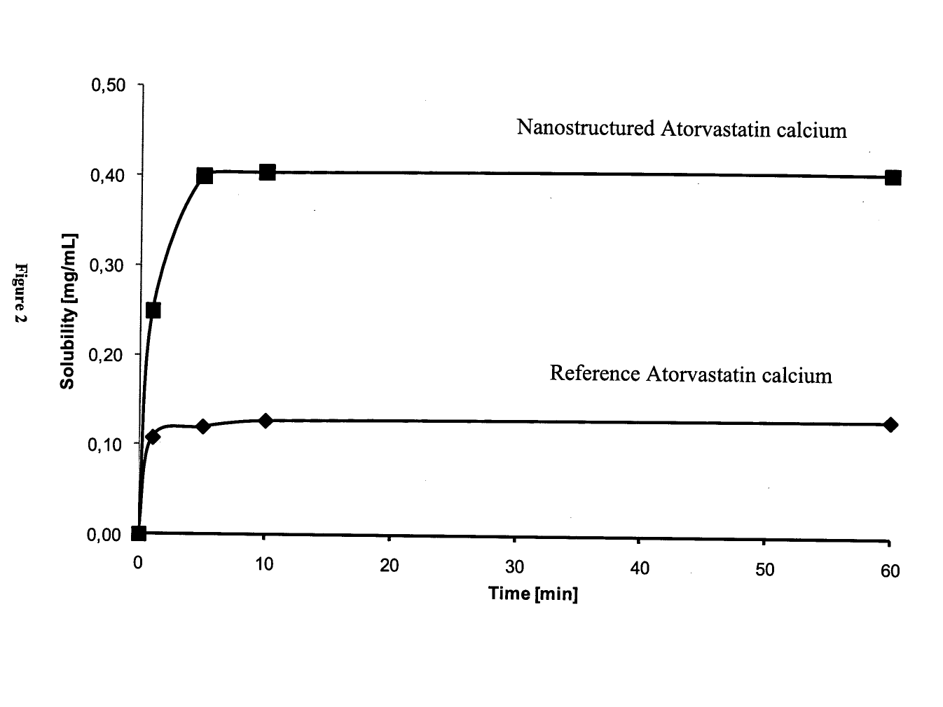

FIG. 2: Comparative Dissolution Test of Reference Atorvastatin Calcium and Nanostructured Atorvastatin Calcium

### EXAMPLE 3

Proton Nuclear Magnetic Resonance Measurements

In order to support the increased solubility of the nanostructured Atorvastatin calcium H-NMR investigations were performed. The Proton Nuclear magnetic resonance (¹H-NMR), 2D ¹H-NOESY and DOSY measurements were performed on Bruker DRX 400 MHz NMR instruments. Nanostructured Atorvastatin calcium, physical mixture of Atorvastatin calcium and stabilizers and placebo prepared in the same way as the nanostructured Atorvastatin calcium in absence of Atorvastatin calcium were in investigated in D₂O solution and H-NMR spectra were recorded. The reference Atorvastatin Calcium could not be investigated due its water insolubility.

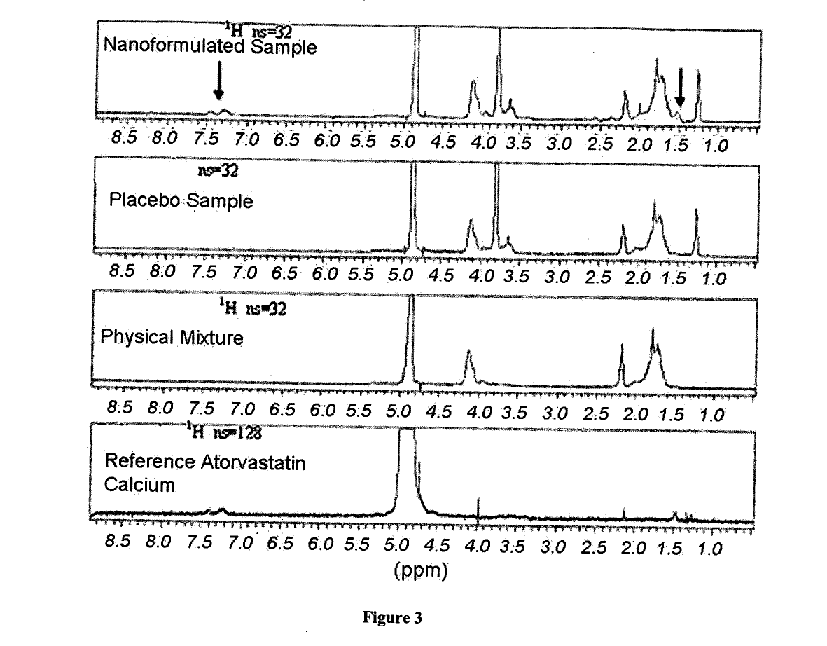

The spectrum of the placebo sample was identical to the spectrum of the stabilizer (PVA). Comparing the spectra of the nanostructured Atorvastatin Calcium to the placebo, two differences could be observed at 1.52 ppm and 7.25-7.5 ppm intervals. These peaks could not be identified in the spectra of the placebo and physical mixture; these characteristic peaks belong to the nanosized Atorvastatin Ca (FIG. 3.). It can be concluded that the nanonization resulted in higher solubility in contradiction to the physical mixture where the presence of the stabilizer could not increase the solubility of the Atorvastatin calcium.

FIG. 3. H-NMR Spertra

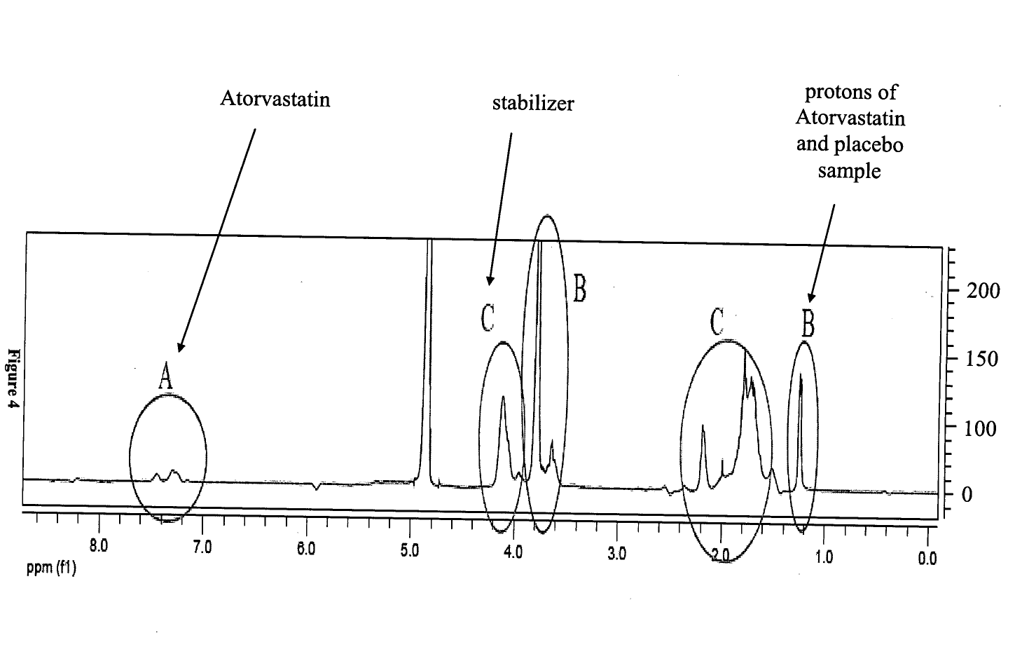

The diffusion coefficient can be calculated from the results of the DOSY measurements (FIG. 4.):

Nanostructured Atorvastatin Ca: D≈1.58* 10⁻¹⁰ m²/s

Placebo: D≈3.72*10⁻¹⁰ m²/s

Stabilizer D≈1.81*10⁻¹⁰ m²/s

Water: D≈9.4*10⁻¹⁰ m²/s

FIG. 4. DOSY Spectra

The diffusion coefficient of the nanostructured Atorvastatin calcium is higher than the diffusion coefficient of the PVA (stabilizer) indicating the nanostructured particle formation and the presence of “free” Atorvastatin in the solution.

2. Instantaneous Wettability and Redispersibility of Nanostructured Atorvastatin

For the Atorvastatin to dissolve, its surface has first to be wetted by the surrounding fluid. The nanostructural forms possess a chemically randomized surface which expresses hydrophobic and hydrophilic interactions due to the nature of the stabilizer/(s) and active pharmaceutical ingredient, which can lead to improved wettability. If the surface of the Atorvastatin nanoparticles of the invention is functionalized by hydrophilic groups/stabilizer(s), a higher degree of hydrophility causes faster surface wetting and faster dissolution compared to the original crystalline form. This advanced property of the Atorvastatin nanoparticles of the present invention is supported by the results of the redispersibility test also. Due to the bigger surface area of the nanostructured Atorvastatin base and its pharmaceutically acceptable salts and the hydrophilic groups of the stabilizer(s) the surface wetting is instantaneous compared to the crystalline forms.

### EXAMPLE 3

Visual Observation of Nanostructured Atorvastatin Calcium's Wettability

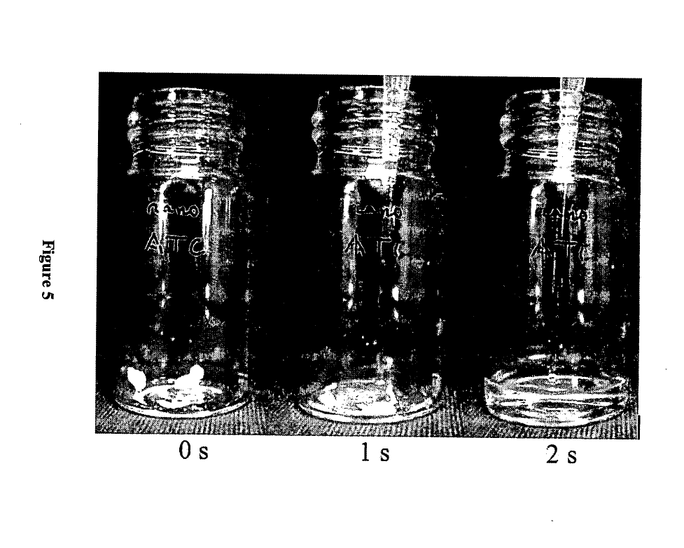

The wettability of nanostructured Atorvastatin calcium powder was performed by dispersing 1 mg nanosized Atorvastatin calcium powder in 0.4 mL distillate water. Following the distillate water addition the vial was gently shaken by hand resulting colloid dispersion of nanostructured Atorvastatin calcium particles as it is demonstrated in FIG. 5.

FIG. 5 Instantaneous Redispersibility and Wettability of Nanostructured Atorvastatin Calcium in Distillate Water

An additional feature of the nanostructured Atorvastatin or its pharmaceutically acceptable salts compositions of the present invention is that the dried nanoparticles stabilized by stabilizer(s) can be redispersed instantaneously or by the addition of traditional redispersants such as mannitol, sucrose.

### EXAMPLE 4

Redispersibility Test of Nanostructured Atorvastatin Calcium

Redispersibility test was performed by redispersing nanostructured Atorvastatin calcium powder in distillate water. 5.4 mg freeze dried nanostructured Atorvastatin calcium was redispersed in 2 mL distillate water under vigorous stirring. The particles size of the redispersed sample was measured by DLS method (Nanotrac instrument, Mictrotrac Co., USA).

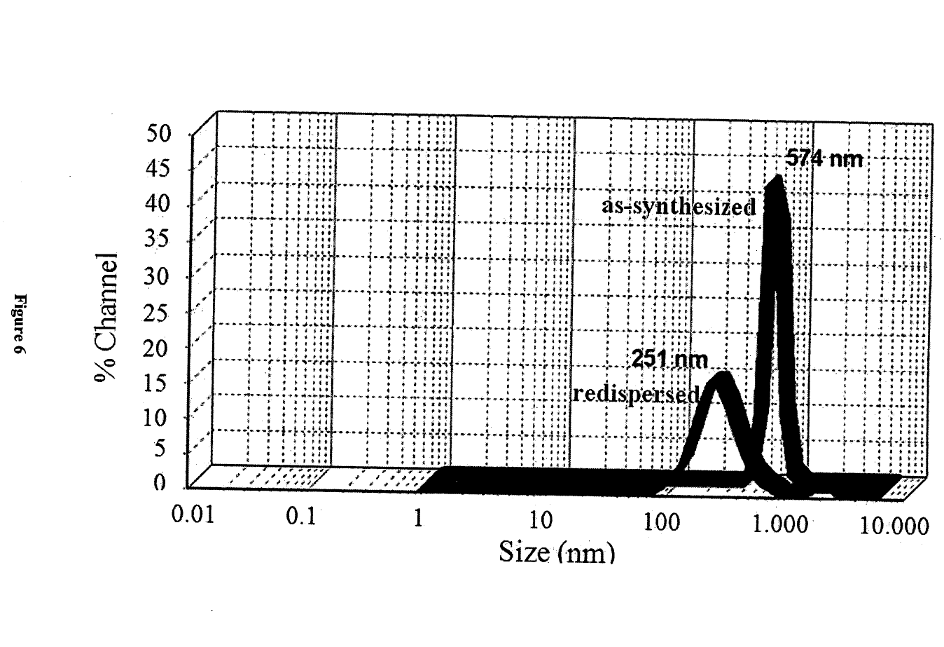

The mean particle size of redispersed nanostructure Atorvastatin calcium (intensity-based average) is d=251 nm, while d(90) value is 492 nm as demonstrated in FIG. 6.

The significant benefit which can be obtained by nanoformulation is that the Atorvastatin calcium nanoparticles of the present invention can be redispersed after the drying/solid formulation procedure having similar average particle size. Having the similar average particles size after the redispersion, the dosage form cannot lose the benefits afforded by the nanoparticle formation. A nanosize suitable for the present invention is an average particle size of less than about 600 nm.

FIG. 6: Size and Size Distribution of the Atorvastatin Calcium the Nanoparticles Before (As-Synthesized) and after its Redispersion.

3. Crystallographic Structure of Nanostructured Atorvastatin or its Pharmaceutically Acceptable Salts Compositions of the Invention

The chemical stability of solid drugs is affected by the crystalline state of the drug. Many drug substances exhibit polymorphism. Each crystalline state has different chemical reactivity. The stability of drugs in their amorphous form is generally lower than that of drugs in their crystalline form, because of the higher free-energy level of the amorphous state. Decreased chemical stability of solid drugs brought about by mechanical stresses such as grinding is to a change in crystalline state. The chemical stability of solid drugs is also affected by the crystalline state of the drug through differences in surface area. For reaction that proceeds on the solid surface of the drug, an increase in the surface area can increase the amount of drug participating in the reaction.

It was surprisingly found that the controlled nano-precipitation of Atorvastatin in the presence of the selected stabilizer(s) resulted in stable amorphous nanostructured Atorvastatin particle formation which can be characterized by increased solubility and dissolution rate compared to the reference API.

### EXAMPLE 5

Crystallographic Structure Determination by Powder X-ray Diffraction Analysis

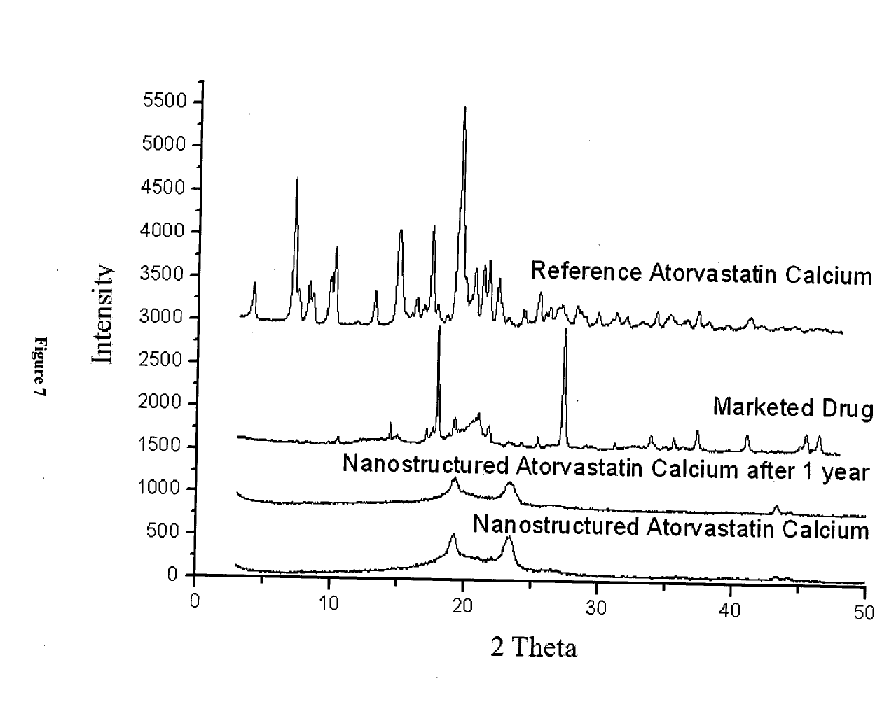

The structure of the Atorvastatin calcium nanoparticles was investigated by X-ray diffraction analysis (Philips PW1050/1870 RTG powder-diffractometer). The measurements showed that the nanostructured Atorvastatin calcium compositions are amorphous. The X-ray diffractograms are demonstrated in FIG. 7. The stability of the amorphous nanostructured Atorvastatin calcium was monitored over a year. As it is demonstrated in FIG. 7. no structural change (crystallization) was observed during one year storage.

FIG. 7 X-ray Diffractograms of Reference Atorvastatin Calcium, Nanostructured Atorvastatin Calcium of the Invention and Marketed Drug

### EXAMPLE 6

Crystallographic Structure Determination by Raman Spectroscopy

The structure of the Atorvastatin calcium nanoparticles was also investigated by Raman spectroscopy. Raman studies were performed by using a Yobin-Yvon/Horiba micro-Raman Spectrometer (Model: Labram) equipped with a 532 nm Nb-YAG laser. Spectra collection was performed at room temperature under the following conditions: 50× microscope objectives, with a D 0.6 filter, accumulation time was 10 s and the scan number was about 4 (these parameters were dependant on the sample). The spectrum recording was preformed with a CCD detector.

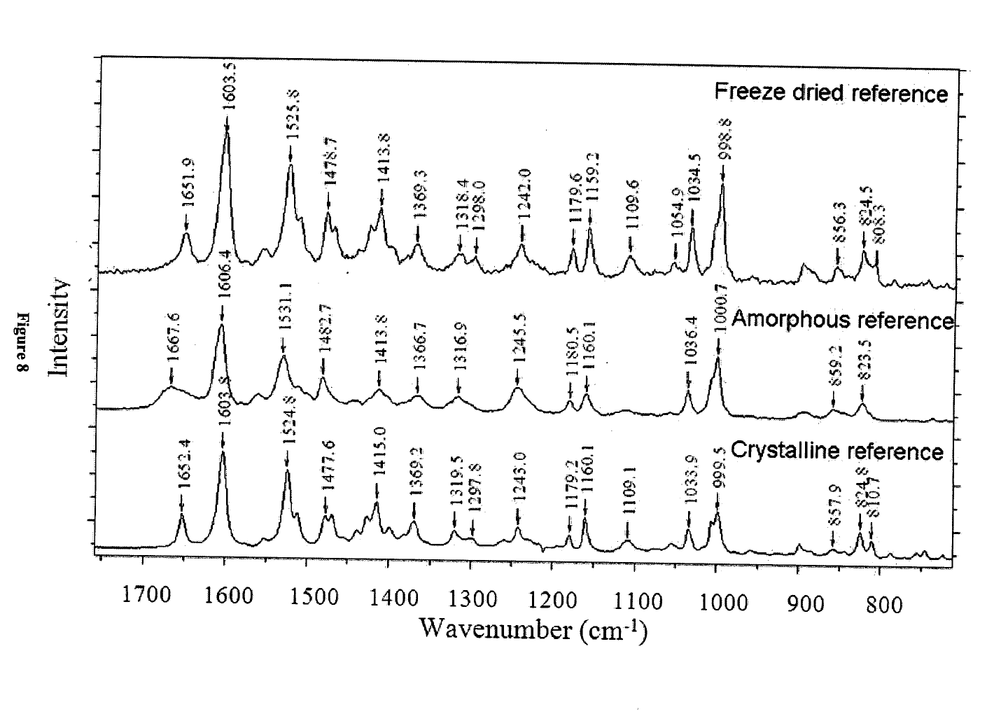

In FIG. 8. the Raman spectra of amorphous Atorvastatin Calcium (Márcia C. Breitkreitz et.al., II Latin-American Symposium on Polymorphism and Crystallization in Drugs and Medicines, Min-Soo Kim et.al, European Journal of Pharmaceutics and Biopharmaceutics 69 (2008) 454-465), freeze dried Atorvastatin Calcium produced by precipitation and crystalline Atorvastatin Calcium are shown. The freeze dried reference produced by precipitation cannot be considered as amorphous material, its characteristic bands show similarity with the crystalline reference API, in some cases broader bands can be indentified in the spectrum of the freeze dried reference API which could refer to the presence of amorphous phase. However pure amorphous Atorvastatin Calcium cannot be produced by simple precipitation method as it is seen from the results.

FIG. 8: Raman Spectra of Amorphous Atorvastatin Ca, Freeze Dried Atorvastatin Ca Produced by Precipitation and Crystalline Atorvastatin Ca

Comparing the Raman spectrum of nanostructured Atorvastatin Calcium to the spectra of the amorphous and crystalline reference APIs, it can be seen that the characteristic bands of the nanostructured Atorvastatin Ca in the spectrum show similarity with the characteristic bands of the amorphous API, so the nanostructured Atorvastatin Calcium could be considered as amorphous material. The amorphous structure of the nanosized Atorvastatin Calcium can be supported by the results of the XRD measurements seen in FIG. 7.

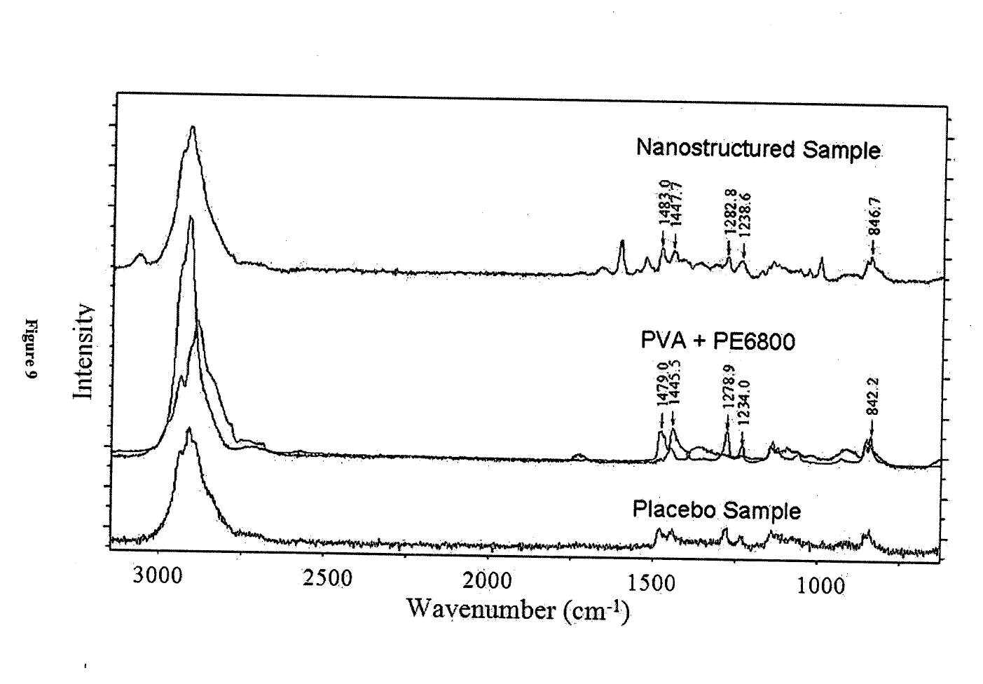

The characteristic Raman band of the stabilizer (Pluronic PE 6800) can be identified in the Raman spectrum of the nanostructured Atorvastatin Calcium below 1500 cm⁻¹ (FIG. 9.). The Raman bands of the stabilizer (Pluronic PE 6800) show 4-5 cm⁻¹ band shift in case of the nanostructured Atorvastatin Calcium considering these Raman bands as characteristic band for the nanostructured Atorvastatin calcium.

FIG. 9: Characteristic Raman Bands of the Stabilizers

### EXAMPLE 7

Crystallographic Structure Determination by Infrared Spectroscopy

Micro Raman spectroscope with ATR module equipped with infrared light-source (diode laser) was used for the investigation. The detector was a MCT (mercury—cadmium—tellurium) detector. The instrument was equipped with different objectives 10× (for optical imaging), ARO (for diffuse reflection measurement) and ATR (for attenuated total reflection measurement). Spectra could be detected in the range of 4000-650 cm⁻¹.

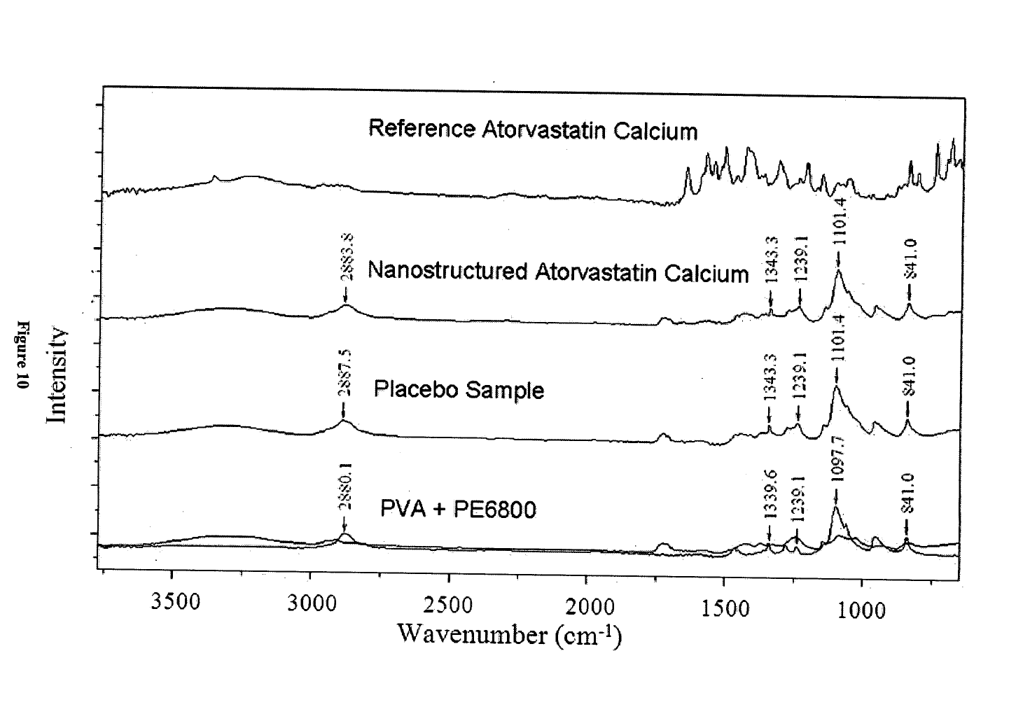

Comparing the spectrum of the nanosized Atorvastatin Calcium to the spectrum of the placebo sample (FIG. 10.), at 2887 cm⁻¹ a band shift can be identified which is a characteristic band of the CH vibration of the stabilizer (Pluronic: block copolymer of ethylene oxide and propylene oxide). The band shift could indicate the amorphous nanostructured particle formation of Atorvastatin calcium of the present invention.

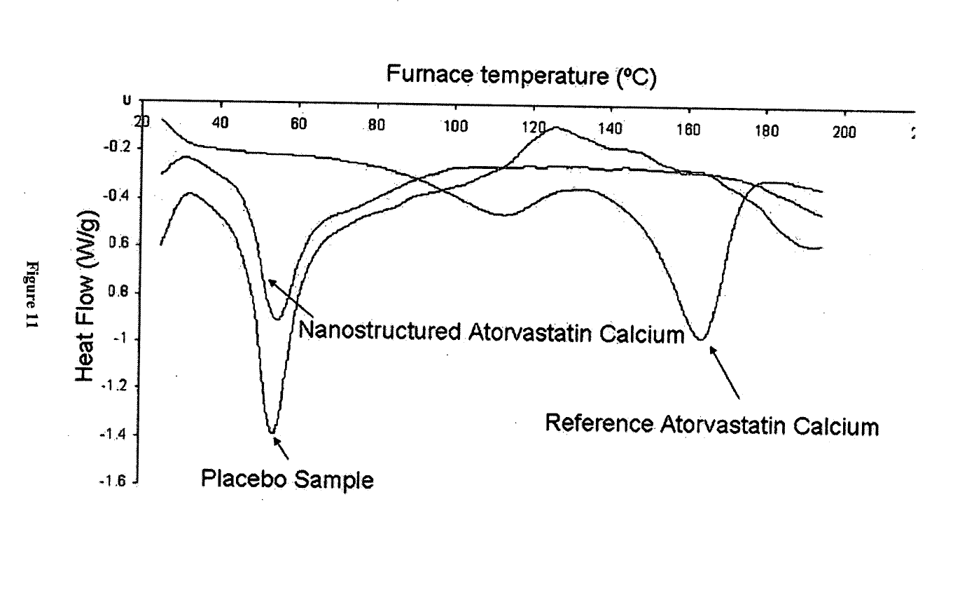

In the ATR spectrum of the placebo sample a few difference can be observed compared to the spectra of the pure stabilizers (Pluronic and PVA) indicating the nanostructured particle formation of Atorvastatin calcium of the present invention also (FIG. 11). These characteristic band of the amorphous nanostructured Atorvastatin calcium of the present invention could be identified in the at 1339 cm⁻¹ and at 1097 cm⁻¹.

FIG. 10: ATR spectra of Atorvastatin Ca (Reference), Nanosized Atorvastatin Ca, Placebo and Stabilizers

### EXAMPLE 8

Crystallographic Structure Determination by Differential Scanning Calorimetry

DSC92 calorimeter (Setaram) was used for the investigation. The thermograms were run at a scanning of 10° C./min, from 25 to 400° C., in nitrogen atmosphere (1.6 bar), approximately 10 mg sample was measured.

The characteristic sharp melting peak of the crystalline reference Atorvastatin Calcium cannot be identified on the thermogram of the nanostructured Atorvastatin Calcium indicating the amorphous character of the nanostructured Atorvastatin calcium as seen in FIG. 11. Endothermic heat effect can be observed at 55° C. on the thermograms of the placebo and nanostructured Atorvastatin calcium which could be explained by the water loss.

Above 100° C., baseline shifts can be observed on the thermogram of the placebo which is missing from the thermogram of the nanostructured Atorvastatin Calcium. This could be explained by the water loss of the hygroscopic stabilizers or could refer to a nanostructured matrix formation in the presence of Atorvastatin Ca.

FIG. 11: DSC Thermograms of the Reference API, Nanostructured Atorvastatin Calcium and Placebo

4. Equivalent Bioavailability of the Nanostructured Atorvastatin Calcium

The amorphous nanostructured Atorvastatin or its pharmaceutically acceptable salts compositions of the invention are proposed to exhibit bioequivalent bioavailability compared to the marketed drug.

### EXAMPLE 9

Comparative in vivo Pharmacokinetic Test on Female Beagle Dogs in Fasted Condition

This study was designed to compare the relative oral bioavailability of two Atorvastatin formulations:
Test formulation: nanostructured Atorvastatin calcium formulation measured into wafer capsule for administration
Reference formulation: commercially available Sortis 40 mg tablet (administered in wafer capsule) manufactured by Pfizer AG.

Experimental Protocols

Comparative in vivo pharmacokinetic test was a cross-over, single dose, two period study. Six female Beagle dogs received a single oral dose of the test and the reference formulations containing the same amount of Atorvastatin using 4-week washout period. The dose of the active ingredient was 40 mg/animal. The plasma concentrations of Atorvastatin were quantified using a reliable bioanalytical method.

To characterize the systemic exposure of Atorvastatin the main pharmacokinetic parameters (Cₘₐₓ, Tₘₐₓ, t_{1/2λ} and AUC) were determined for the individual plasma level versus time curves. The parameters obtained after administration of the test formation were compared to those obtained for the reference tablet.

It is known from the literature (Drugs 2001, 61 (12): 1835-1881) that following oral administration of Atorvastatin under fed condition the peak concentration (Cₘₐₓ) was reduced and Tₘₐₓ was delayed, yet systemic exposure (AUC) did not show significant changes. Thus, the bioavailability of the two formulations was compared only under fasted condition.

Animals

The Beagle dog is a suitable non-rodent species for pharmacokinetic studies and is acceptable to regulatory authorities. The dog is readily available, easy to handle, house and dose and suitable for investigation of the whole plasma level curve in each individual animal. The systemic exposure was investigated in six dogs. This group size was necessary because of the expected relatively high inter-individual variability due to the first pass effect.

The study was conducted according to the Guide for the Care and Use of Laboratory Animals, NRC, 1996 and in compliance with the principles of the Hungarian Act 1998: XXVIII. regulating animal protection.

Food and Feeding

The animals received sniff Hd-H diet for dogs produced by Sniff, Spezialdiäten GmbH. The food was offered daily 300 g/dog approximately at the same time. The next morning the remaining food was taken away. Before the administrations, the animals were fasted overnight (at least 12 h). On the treatment day, the animals received food approx. 4 hours after the administration.

Blood Collection and Plasma Separation

For determination of plasma levels of Atorvastatin approximately 3 ml of blood was collected in plastic vials with lithium heparin as anticoagulant. The time points of blood collection were the following in both periods: pre-dose (0 min), 15 min, 30 min, 45 min, 1 h, 2 h, 4 h, 6 h, 8 h, 24 h and 48 h after dosing.

Blood was withdrawn the v. cephalica antebrachii or v. saphena with sterile, disposable needles.

After sampling, the blood was kept cooled on crushed ice until centrifugation. Plasma samples were prepared by centrifugation of the blood at 2,000 g for 10 minutes at 4° C. within 60 minutes after blood sampling. The separated plasma (approx. 1 ml) was transferred into Eppendorf tubes. Plasma samples were immediately frozen and stored in deep-freezer (−20±5° C.) until analysis.

The concentrations of Atorvastatin were determined using a reliable chromatographic bioanalytical method.

Pharmacokinetic Evaluation

The pharmacokinetic evaluation was performed by using WinNonlin Professional Version 4.0.1 software (Pharsight Corporation, USA). The individual plasma levels versus time curves were evaluated using a non compartmental method.

Results

The absorption process showed high inter-individual variability resulting in peak concentration between 1-6 hours. The peak concentrations were 7.87±4.46 ng/ml and 9.47±4.96 ng/ml for the reference and the test formulations, respectively. It is noticeable, that the blood sampling schedule was not optimal for precise determination of the peak concentrations occurred at unexpectedly late time-points.

Following the peak the plasma levels decreased fast in relation to time. For the reference formulation the concentrations could be quantified until 8 hours post-dose in 5 dogs. Only in Dog 2 the last measurable plasma level was found at 24 hours. For the test formulation Atorvastatin could be quantified until 24 hours in 2 dogs and until 48 hours in 1 dog.

The first decrease of the plasma level curves could be characterized with a short elimination half-life. For those dogs, for which the plasma levels were quantifiable only at 8 hours post-dose the elimination half-lives were 1.2-1.7 hours. The second compartment and the corresponding slower half-lives (5-11 hours) could be detected only if the concentrations remained measurable until 24-48 hours post-dose.

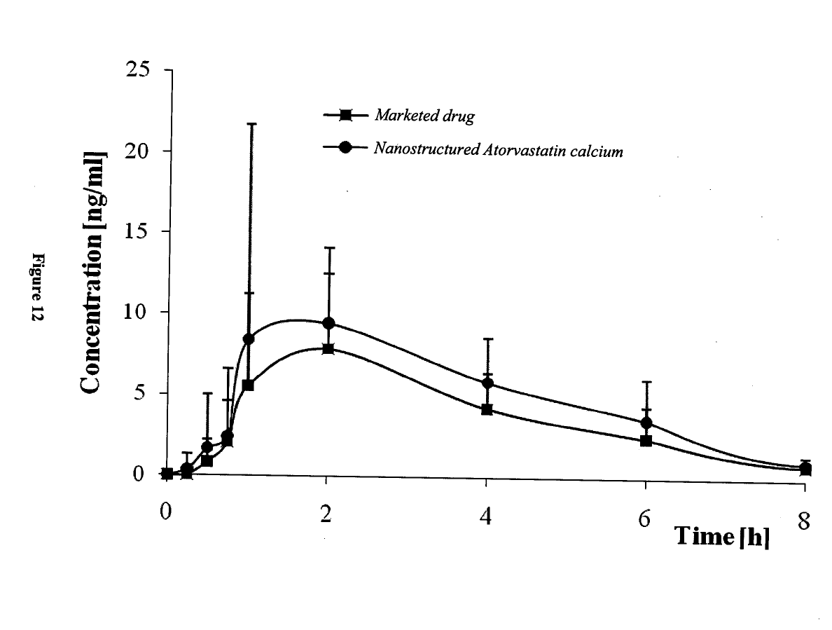

The total systemic exposure was characterized by the area under the curve values. The AUCₗₐₛₜ values were 38.05±8.34 ng*h/ml and 51.90±8.99 ng*h/ml for the reference and the test formulations, respectively (FIG. 12).

FIG. 12: Serum Concentrations of Atorvastatin After Oral Administration of 40 mg Nanostructured Atorvastatin and Reference Test Substance

Both the mean peak concentration and the total exposure were somewhat higher following administration of the test formulation than after dosing of Sortis tablet. The relative bioavailability (calculated from the AUCₗₐₛₜ values) of the test formulation was 136%+/−20% (FIG. 3). Given the high inter individual differences in the plasma concentration time curves this can basically be considered bioequivalent.

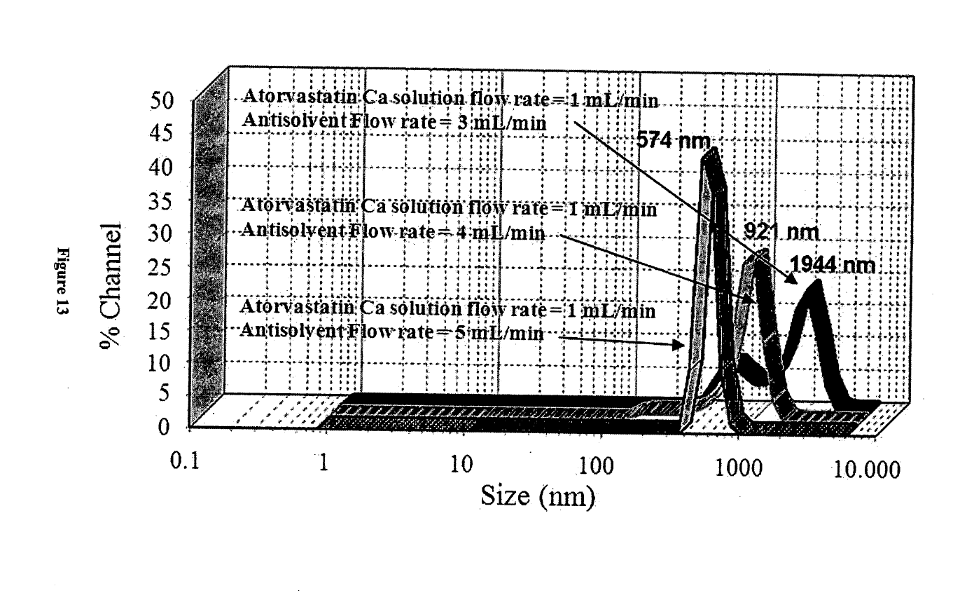

FIG. 13: Main Pharmacokinetic Parameters of Atorvastatin in Fasted Female Dogs

B. Compositions

The nanoparticles of Atorvastatin, its pharmaceutically acceptable salts and compositions of them according to the invention have an average particle size of less than about 600 nm. The stable amorphous nanostructured particles of the present invention are characterized by increased solubility and bioequivalent biological performance compared to the marketed crystalline drug.

The stabilizers preferably are associated or interacted with the Atorvastatin and its pharmaceutically acceptable salts, but do not chemically react with the Atorvastatin or themselves.

The nanoparticles of Atorvastatin, its pharmaceutically acceptable salts and co-crystals of the invention can be prepared by solvent-antisolvent nano-precipitation methods using stabilizer(s).

Particle Size of the Nanostructured Atorvastatin and its Pharmaceutically Acceptable Salts Particles

The invention contains Atorvastatin and its pharmaceutically acceptable salts nanoparticles, which have an average particle size of less than about 600 nm as measured by dynamic light scattering method.

By “an average particle size of less than about 600 nm” it is meant that at least 50% of the Atorvastatin base and its pharmaceutically acceptable salts have a particle size of less than the average, by number/intensity, i.e., less than about 600 nm, etc., when measured by the above-noted technique.

### EXAMPLE 10

Process for Producing Nanostructured Atorvastatin Calcium

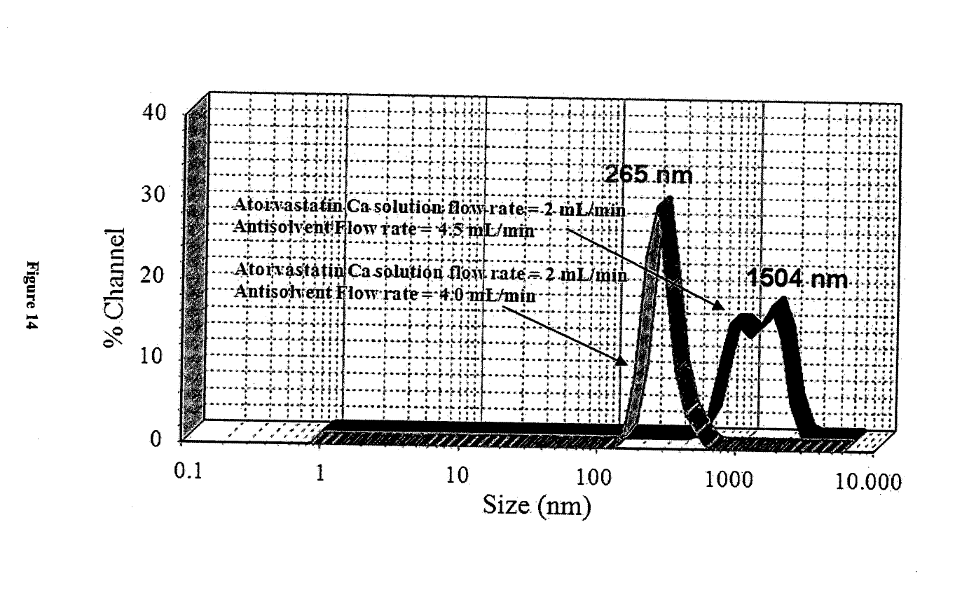

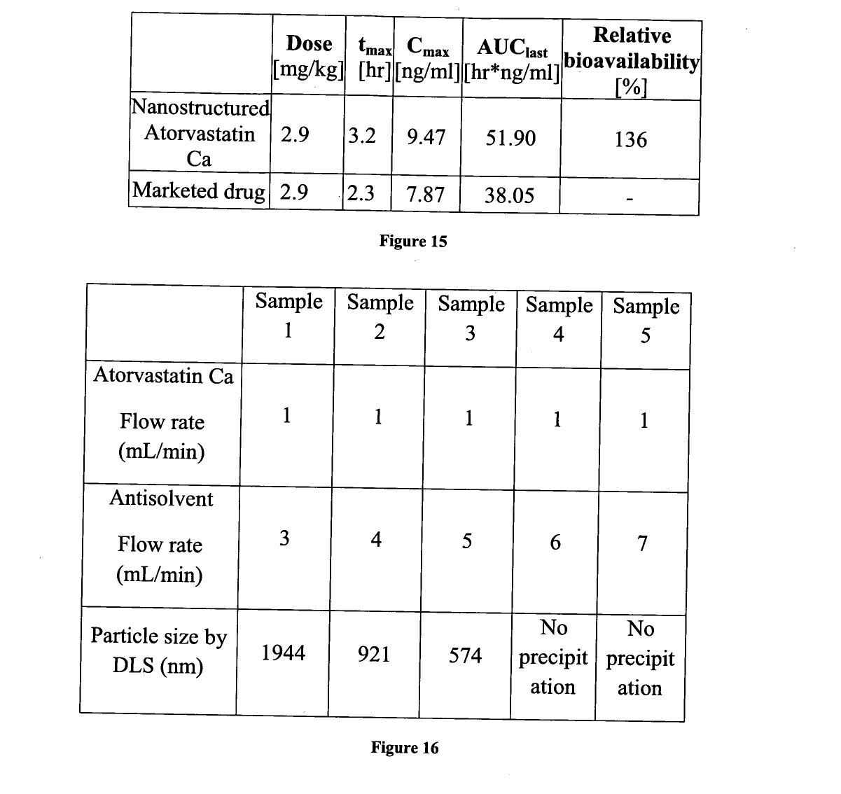

During the experiments Atorvastatin calcium (ATC) nanoparticles were prepared in a microfluidic based continuous flow reactor. As a starting solution, 500 mg Atorvastatin calcium and 1000 mg Pluronic PE6800 dissolved in 50 mL methanol was used. The prepared solution was passed into the reactor unit with 1 mL/min flow rate using a feeding unit. Meanwhile, using a second feeding unit, a solution of 2000 mg poly(vinylalcohol) (Mw=30.000-70.000) dissolved in 400 mL distilled water was passed into a mixing unit with 5 mL/min flow rate, where it was mixed with the solution containing Atorvastatin calcium coming from the first reactor unit. The nanoparticles are continuously produced at atmospheric pressure due to the precipitation by poly(vinylalcohol) solution passed into the mixing unit. The produced colloidal solution driven through the second reactor unit gets to the dynamic light scattering unit (Nanotrac) integrated to the device, which can detect the particle size of the obtained nanoparticle continuously. The size of the nanoparticles can be controlled in wide range by changing the flow rates (see FIG. 14.). The mean particles size of the Atorvastatin calcium particle was 574 nm in the best case (see FIG. 15). Changing the flow rates the particles size can be varied from 574 up to 1944 nm.

FIG. 14: Particle Size and Size Distribution of Atorvastatin Calcium Nanoparticles Using Different Flow Rates.

FIG. 15: Effect of the Flow Rates on the Particle Size of Atorvastatin Calcium

### EXAMPLE 11

Process for Producing Nanostructured Atorvastatin

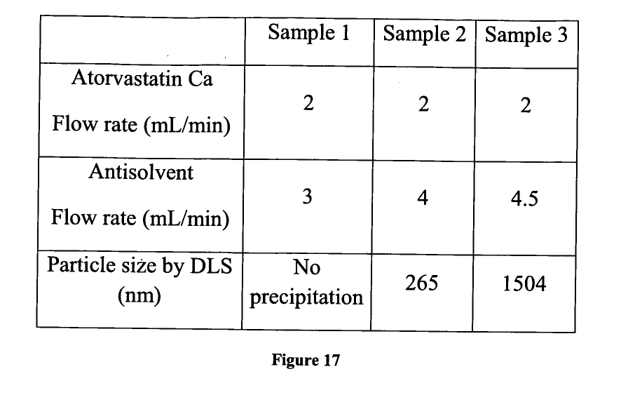

During the experiments Atorvastatin nanoparticles were prepared in a microfluidic based continuous flow reactor. As a starting solution, 200 mg Atorvastatin Calcium and 600 mg poly(vinylpirrolidone) (PVP 10) dissolved in 40 mL distilled EtOH was used. The prepared solution was passed into the reactor unit with 2 mL/min flow rate using a feeding unit. Meanwhile, using a second feeding unit, a solution of 50 mg sodium dodecylsulfate (SDS) dissolved in 100 mL 0.1 M HCl solution was passed into a mixing unit with 4 mL/min flow rate, where it was mixed with the solution containing Atorvastatin Calcium coming from the first reactor unit. The nanoparticles are continuously produced at atmospheric pressure due to the precipitating effect of 0.1 M HCl solution passed into the mixing unit. The produced colloidal solution driven through the second reactor unit gets to the dynamic light scattering unit (Nanotrac) integrated to the device, which can detect the particle size of the obtained nanoparticle continuously. The size of the nanoparticles can be controlled in wide range by changing the flow rates (see FIG. 16.). The particles size of the Atorvastatin particle was 265 nm in the best case (see FIG. 17). Changing the flow rates the particles size can be varied from 265 up to 1504 nm.

FIG. 16: Particle Size and Size Distribution of Atorvastatin Nanoparticles Using Different Flow Rates

FIG. 17: Effect on the Flow Rates on the Particle Size of Atorvastatin

1-11. (canceled)

12. A stable nanostructured Atorvastatin composition comprising:
(a) nanostructured Atorvastatin or its pharmaceutically acceptable salt having an average particle size of less than about 600 nm;
(b) at least one stabilizer,
wherein the composition is prepared in a continuous flow reactor.

13. The composition according to claim 12, wherein the average particle size is between 600 nm and 50 nm, preferably 400 nm and 50 nm, preferably 300 nm and 50, and the stabilizer is selected from the group of non-ionic, anionic, cationic polymers/surfactants, and zwitterionic surfactants, or combinations thereof.

14. The composition according to claim 12 wherein the stabilizer is selected from the group of cellulose and its derivatives, polyvinylpyrrolidone, sodium lauryl sulfate, gelatin, cetostearyl alcohol, polyethylene glycols, acetic acid ethenyl ester polymer with 1-ethenyl-2-pyrrolidinone (PVP/VA copolymers), sodium dodecyl benzene sulfonate, tocopheryl 5 polyethylene glycol succinates, urea, citric acid, sodium-acetate, polyethoxylated castor oils and its derivateives, polyoxyethylene stearates, methylcellulose, hydroxyethylcellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide); poloxamines 10 (e-g) Tetronic, also known as Poloxamine, which is a tetrafunctional block copolymer derived from sequential addition of propylene oxide and ethylene oxide to ethylenediamine, D-alfa-Tocopherol polyethylene glycol 1000 succinate, poly(2-ethyl-2-oxazoline), poly(methyl vinyl ether), random copolymers of vinyl pyrrolidone and vinyl acetate, such as Plasdone S630.

15. The composition according to claim 12 wherein the stabilizer is selected from the group of polyvinyl alcohol, poloxamers, polyvinylpyrrolidone, sodium acetate, sodium lauril sulfate.

16. A process for the preparation of nanostructured Atorvastatin composition salt according to claim 12, comprising precipitating nanostructured Atorvastatin or its pharmaceutically acceptable salt from a solution of Atorvastatin or its pharmaceutically acceptable salt and at least one stabilizer with other solvent, preferably water containing optionally at least one stabilizer and optionally in the presence of a pharmaceutically acceptable acid in a continuous flow reactor, preferably in a microfluidic based conterminous flow reactor.

17. The process according to 16, comprising (1) dissolving Atorvastatin or its pharmaceutically acceptable salt and at least one stabilizer in a suitable solvent; (2) adding the solution from step (1) to a solution of a pharmaceutically acceptable acid; and (3) precipitating the formulation from step (2).

18. The process according to claim 17, wherein the pharmaceutically acceptable acid is hydrochloride acid, acetic acid, citric acid, maleic acid, oxalic acid, formic acid or, benzoic acid, preferably acetic acid.

19. A pharmaceutical composition comprising a nanostructured composition according to claim 12 together with pharmaceutically acceptable auxiliary materials.

20. A pharmaceutical composition according to claim 19, wherein the composition is formulated into a dosage form preferably in the form of oral, pulmonary, rectal, parenteral, intracisternal, intravaginal, intraperitoneal, ocular, otic, local, buccal, nasal or topical administration.

21. Use of nanostructured composition according to claim 12 for preparation of a medicament.

22. Use of nanostructured composition according to claim 12 for the treatment of high blood cholesterol level, stabilizing plaque or preventing stroke.

23. A method of treating a subject in need thereof by administering to the subject an effective amount of nanostructured Atorvastatin composition according to claim 12.

24. The method according to claim 23, wherein the composition has
a solubility at least about 0.4 mg/ml in water,
instantaneous redispersibility in physiological mediums,
at least equal/bioequivalent absorption compared to the marketed form in human gastrointestinal tract,
faster onset of action,
for decreasing the dosage used.

25. The composition according to claim 12, wherein the composition is prepared in a microfluidic based continuous flow reactor.
